(19) United States
(12) Patent Application Publication
(10) Pub. No.: US 2017/0360922 A1
(43) Pub. Date: Dec. 21, 2017

(54) NUTRITIONAL SUPPLEMENTS AND METHODS FOR PRIMARY PREVENTION OF FOOD ALLERGIES

(71) Applicant: Paul Turke, Whitmore Lake, MI (US)

(72) Inventor: Paul Turke, Whitmore Lake, MI (US)

(21) Appl. No.: 15/628,729

(22) Filed: Jun. 21, 2017

Related U.S. Application Data
(60) Provisional application No. 62/352,738, filed on Jun. 21, 2016.

(51) Int. Cl. A61K 39/35; A61K 39/00

(52) U.S. Cl. CPC A61K 39/35; A61K 2039/545

(57) ABSTRACT

The present invention relates to formulations and methods for preventing food allergies in a subject. Embodiments of the invention provide for administration of allergens from eight types of food that cause food allergies in western societies: peanut, tree nuts, milk, egg, soy, wheat, fish, and shellfish. The allergens may be provided directly to the subject, or to the subject's mother, who may be pregnant or nursing.

### CROSS-REFERENCE TO RELATED APPLICATION

This application claims the benefit of U.S. Provisional Application No. 62/352,738, filed Jun. 21, 2016, which is incorporated by reference in its entirety.

### BACKGROUND OF THE INVENTION

In recent years, the number of individuals experiencing food allergies has drastically increased: childhood food allergies have increased by about 50% in the past two decades in the United States, with peanut allergies increasing from an occurrence in 0.4% of children in 1997 to 1.4% today. Consumption of, or even simple exposure to, triggering foods can provoke an immune response, sometimes severe, which can present in a number of ways, including but not limited to inflammation, gastrointestinal distress, cramping, nausea, and difficulty breathing. At a minimum, such allergies can cause discomfort; in severe cases, reactions can include anaphylactic shock, and may even be fatal.

Mitigation of food allergies typically has focused on treatment and prevention once the symptoms arise later in life. The conventional wisdom has been for pregnant and nursing mothers and young children to avoid consuming foods known to trigger allergic reactions. Eight types of food account for approximately 90% of food allergies: peanuts, tree nuts, dairy (cow's milk), wheat, eggs, soy, fish, and shellfish (crustaceans.) According to the conventional wisdom, by avoiding exposure to allergens, pregnant and nursing mothers and young children avoid a potentially catastrophic allergic reaction.

As the prevalence of food allergies has become an important public health problem, there is a continuing need to develop new strategies for prevention.

### SUMMARY OF THE INVENTION

In one embodiment, the present invention relates to a nutritional supplement formulation including therapeutically effective amounts of a first allergen, a second allergen, a third allergen, and a pharmaceutically acceptable carrier. The first allergen, the second allergen, and the third allergen may be selected from the group consisting of a peanut allergen, a milk allergen, a wheat allergen, a tree nut allergen, an egg allergen, a soy allergen, a fish allergen, and a shellfish allergen in any combination. The supplement may also contain one or more of a fourth allergen, a fifth allergen, a sixth allergen, a seventh allergen, and an eighth allergen, each of which may be chosen from the aforementioned group.

In another embodiment, the present invention relates to a method of preventing a food allergy in a subject, comprising administering therapeutically effective amounts of a first, second, and third allergen to the subject's mother. The mother may be pregnant at the time of administration, or the mother may be breastfeeding at the time of administration.

In a further embodiment, the present invention relates to a method of preventing food allergies in a subject comprising administering therapeutically effective amounts of a first, second, and third allergen to the subject. Administration may be conducted orally. The subject may be from about 3 months to about 4 years old during the period of administration.

### DETAILED DESCRIPTION

The terms “allergen” and “antigen” are used interchangeably and refer to a substance which can provoke an immune response when an individual ingests or is otherwise exposed to said substance. An allergen or antigen may be a compound or class of compounds, a mixture of compounds, a mixture made up of one or more cells, or any other substance capable of inducing an immune response. An allergen or antigen may be a protein, a non-protein, a polysaccharide, a lipid, a small molecule, or any other substance capable of inducing an immune response. An allergen or antigen may be obtained or isolated from food or may besynthesized from, or derived from, another source. For example, a protein from peanut known to be an allergen which has been recombinantly expressed in a microbe (for instance, a bacterium or a yeast) would be considered a peanut allergen. When administered in accordance with the methods described herein, an “allergen” or “antigen” may be administered in the form of food item(s) containing such allergen or antigen or may be administered in a nutritional supplement formulation as described herein. For example, a peanut allergen may be administered in a variety of forms, including without limitation a whole peanut, peanut butter, a powder created by grinding the seed of a peanut, or a purified protein from peanut.

When used in conjunction with a food type, the term “allergen” or “antigen” means a substance present in said food type which may induce an allergic reaction. For example, a “peanut allergen” refers to a substance present in peanuts which may provoke an immune response. Allergens and antigens may be isolated from their respective food types or may be prepared synthetically.

The term “subject” refers to an individual in whom food allergies are intended to be prevented, preferably a mammal, and most preferably a human. In some cases, the subject may be a human embryo, fetus, infant, toddler, or child. The subject may be from birth to about 3 months old, from birth to about 6 months old, from birth to about 9 months old, from birth to about 12 months old, from birth to about 18 months old, from birth to about 24 months old, from birth to about 30 months old, from birth to about 36 months old, from about 3 months old to about 4 years old, or from about three months old to about 5 years old.

The term “mother” refers to an individual who carries the subject in her uterus or who is nursing (breastfeeding) the subject and is not limited to the biological mother of the subject. For example, the term “mother” includes a woman carrying a child as a surrogate or breastfeeding a child other than her own, as well as the biological mother of the subject.

One embodiment of this invention relates to a nutritional supplement formulation for preventing a food allergy in a subject. The nutritional supplement formulation may include therapeutically effective amounts of allergens or potentially-allergenic substances present in one, two, three, four, five, six, seven, eight, or more food types.

Eight food types commonly responsible for food allergies are peanuts, tree nuts, dairy (milk), wheat (and other gluten-containing grains), soy, egg, fish, and shellfish. Allergens of each type are described below. The nutritional supplement formulations described herein may contain one or more allergens present in these eight food types or may contain one or more other food allergens present in other food types, such as sesame seeds, strawberries, chocolate, celery, mustard, lupin (a legume), kiwi, and foods containing sulfur dioxide (sulfites).

The term “peanut allergens” includes but is not limited to allergens present in the plant Arachis hypogaea, particularly the seed. Proteins of peanut known to cause allergic reaction in certain individuals include Ara h 1, 2, 3, 6, 8, and 9. A purified Ara h protein may be used as an allergen in the nutritional supplements and methods described herein. Whole peanuts, powders of whole peanut seed, or oils pressed therefrom may also be used.

The term “tree nut allergens” includes but is not limited to allergens present in the nuts, seeds, and other portions of the plants that produce Brazil nuts, cashews, hazelnuts, pistachios, macadamia nuts, chestnuts, pecans, walnuts, and pine nuts. Tree nut allergens used in the nutritional supplement formulations and methods described herein may be introduced in various forms, including without limitation, whole tree nuts, purified tree nut proteins, powders of whole nuts or seeds, and oils pressed therefrom, among others.

The term “milk allergens” includes but is not limited to allergens present in milk produced by bovine (cows), sheep, and goats. Proteins of milk known to cause allergic reaction in certain individuals include casein and whey. Various sources of milk allergens, including without limitation milk, dehydrated milk, purified casein proteins, purified whey proteins, or a combination thereof may be used in the formulations and methods described here.

The term “wheat allergens” includes but is not limited to allergens present in plants of the genus Triticum. A protein of wheat known to cause allergic reaction in certain individuals is gluten, although other proteins may also produce an allergic reaction. Wheat allergens used in the nutritional supplement formulations and methods described hereing may be introduced in various forms, including without limitation as purified gluten, powders or flours of wheat, or ther wheat products, such as bread.

The term “egg allergens” includes but is not limited to allergens present in chicken eggs. Purified egg white proteins or purified proteins from egg yolk may be used in the formulations and methods described herein. Whole eggs, and powders made from dehydrated egg whites, egg yolks, or both, may also serve to provide egg allergens for the formulations and methods described herein.

The term “soy allergens” includes but is not limited to allergens present in soybeans. Whole soybeans, purified soy proteins, and oils or powders of soybean may serve to provide soy allergens for the formulations and methods described herein.

The term “fish allergen” includes but is not limited to allergens present in finned or scaly fish. Fish meat, purified allergenic proteins of fish, and powders or oils derived from such fish may serve to provide fish allergens for the formulations and methods described herein.

The term “shellfish allergens” includes but is not limited to allergens present in crustaceans and/or mollusks. Exemplary crustaceans include shrimp, prawns, crayfish, crabs, and lobsters. In addition, shellfish may optionally include non-crustacean organisms, particluarly mollusks including clams, oysters, scallops, and mussels. Shellfish, purified allergenic proteins of shellfish, and powders or oils derived from shellfish (particularly shrimp) may serve to provide shellfish allergens for the formulations and methods described herein.

According to one embodiment of this invention, a nutritional supplement formulation may include therapeutically effective amounts of allergens or potentially-allergenic substances present in three of the eight types of foods commonly responsible for food allergies (listed above). The three allergens are selected independently of one another. The amount of each allergen may be the same as the amount of each other allergen, or may be different. In some embodiments, all three allergens are made by dehydrating or freezing an allergenic precursor and grinding them down to a powder. In other embodiments, one or more of the allergens may be a purified protein, and one or more of the allergens may be a powder or oil of an allergenic substance.

In one example, the first allergen may be a peanut allergen, the second allergen may be a milk allergen, and the third allergen may be a wheat allergen.

According to one embodiment of this invention, a nutritional supplement formulation may include therapeutically effective amounts of allergens or potentially-allergenic substances present in four of the eight types of foods commonly responsible for food allergies. The four allergens are selected independently of one another. The amount of each allergen may be the same as the amount of each other allergen, or may be different. In some embodiments, all four allergens are made by dehydrating or freezing an allergenic precursor and grinding them down to a powder. In other embodiments, one or more of the allergens may be a purified protein, and one or more of the allergens may be a powder or oil of an allergenic substance.

In one example, the first allergen may be a peanut allergen, the second allergen may be a milk allergen, the third allergen may be a wheat allergen, and the fourth allergen is selected from the group consisting of a tree nut allergen, a soy allergen, an egg allergen, a fish allergen, and a shellfish allergen.

According to one embodiment of this invention, a nutritional supplement formulation may include therapeutically effective amounts of allergens or potentially-allergenic substances present in five of the eight types of foods commonly responsible for food allergies. The five allergens are selected independently of one another. The amount of each allergen may be the same as the amount of each other allergen, or may be different. In some embodiments, all five allergens are made by dehydrating or freezing an allergenic precursor and grinding them down to a powder. In other embodiments, one or more of the allergens may be a purified protein, and one or more of the allergens may be a powder or oil of an allergenic substance.

In one example, the first allergen may be a peanut allergen, the second allergen may be a milk allergen, the third allergen may be a wheat allergen, and the fourth and fifth allergens are two selected from the group consisting of a tree nut allergen, a soy allergen, an egg allergen, a fish allergen, and a shellfish allergen.

According to one embodiment of this invention, a nutritional supplement formulation may include therapeutically effective amounts of allergens or potentially-allergenic substances present in six of the eight types of foods commonly responsible for food allergies. The six allergens are selected independently of one another. The amount of each allergen may be the same as the amount of each other allergen, or may be different. In some embodiments, all six allergens are made by dehydrating or freezing an allergenic precursor and grinding them down to a powder. In other embodiments, one or more of the allergens may be a purified protein, and one or more of the allergens may be a powder or oil of an allergenic substance.

In one example, the first allergen may be a peanut allergen, the second allergen may be a milk allergen, the third allergen may be a wheat allergen, and the fourth, fifth, and sixth allergens are three selected from the group consisting of a tree nut allergen, a soy allergen, an egg allergen, a fish allergen, and a shellfish allergen.

According to one embodiment of this invention, a nutritional supplement formulation may include therapeutically effective amounts of allergens or potentially-allergenic substances present in seven of the eight types of foods commonly responsible for food allergies. The seven allergens are selected independently of one another. The amount of each allergen may be the same as the amount of each other allergen, or may be different. In some embodiments, all seven allergens are made by dehydrating or freezing an allergenic precursor and grinding them down to a powder. In other embodiments, one or more of the allergens may be a purified protein, and one or more of the allergens may be a powder or oil of an allergenic substance.

In one example, the first allergen may be a peanut allergen, the second allergen may be a milk allergen, the third allergen may be a wheat allergen, and the fourth, fifth, sixth, and seventh allergens are four selected from the group consisting of a tree nut allergen, a soy allergen, an egg allergen, a fish allergen, and a shellfish allergen.

According to another embodiment of this invention, a nutritional supplement formulation may include therapeutically effective amounts of allergens or potentially-allergenic substances present in all eight of the types of foods commonly responsible for food allergies. The eight allergens are selected independently of one another and represent at least one each of a peanut allergen, a milk allergen, a wheat allergen, a tree nut allergen, a soy allergen, an egg allergen, a fish allergen, and a shellfish allergen. The amount of each allergen may be the same as the amount of each other allergen, or may be different. In some embodiments, all allergens are made by dehydrating or freezing an allergenic precursor and grinding them down to a powder. In other embodiments, one or more of the allergens may be a purified protein, and one or more of the allergens may be a powder or oil of an allergenic sub stance.

Nutritional supplement formulations of this invention may comprise allergens present in one, two, three, four, five, six, seven, or eight of the food types described above, and may optionally include allergens present in one or more other food types, and a pharmaceutically acceptable carrier. Such compositions may optionally comprise an additional therapeutic agent. Such agents include, but are not limited to, an antibiotic, an anti-inflammatory agent, a matrix metalloprotease inhibitor, a lipoxygenase inhibitor, a cytokine antagonist, an anti-cancer agent, an anti-viral agent, a cytokine, a growth factor, an immunomodulator, a prostaglandin or an anti-vascular hyperproliferation compound. In particular, one or more immunomodulators may be employed. In certain embodiments, an immunomodulator may act to stimulate the immune system and in doing so improve the efficacy of administered allergens in preventing food allergies. Specific immunomodulators include.

Moreover, in addition to or in place of food allergens, antigenic proteins that are targeted by the immune system in autoimmune disorders may be included in the formulations described herein or may be administered in the methods described herein. Such proteins may be derived from insulin-producing cells, such as the beta cells of pancreatic islets, which when eliminated by an immune response, results in diabetes. In another example, myelin may be provided in order to prevent immune attack against nerve cells, which may assist in prevention of diseases such as multiple sclerosis and other neurodegenerative diseases.

The term “pharmaceutically acceptable carrier” refers to a non-toxic carrier that may be administered to a patient, together with one or more antigens (as defined above), and which does not affect the immunogenic activity thereof.

Pharmaceutically acceptable carriers that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, wool fat and self-emulsifying drug delivery systems (SEDDS) such as alpha-tocopherol, polyethyleneglycol 1000 succinate, or other similar polymeric delivery matrices.

The term “therapeutically effective amount” refers to an amount effective in inducing tolerance to an antigen in a subject. A therapeutically effective amount will differ based upon the mode of administration; for example, the amount of antigen required for direct administration of the allergen to the subject may be less than the amount required to be ingested by a pregnant or nursing woman. However, these ranges are not exclusive, and may overlap. In some cases, the weight of the mother may impact the therapeutically effective amount. In other cases, the weight of the subject may impact the therapeutically effective amount. In still other cases, the therapeutically effective amount may be independent of the weight of the mother and of the subject. The term “preventing food allergies” refers to eliminating or reducing the duration, frequency, or severity of the allergic response suffered by a subject when the subject is exposed to an allergen. A subject in whom a food allergy has been prevented may be substantially free of allergic reaction to the foods or allergens with which they have been treated. Prevention of the food allergy ideally persists for life, but may persist for at least 50 years, at least 40 years, at least 30 years, at least 25 years, at least 20 years, at least 15 years, at least 10 years, at least 5 years, at least 4 years, at least 3 years, at least 2 years, or at least 1 year.

The nutritional supplement formulations of this invention may be administered orally, but may also be administered by any other route known in the relevant art, including parenterally, by inhalation spray, topically, rectally, nasally, buccally, vaginally or via an implanted reservoir. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the antigens present therein. The term parenteral as used herein includes subcutaneous, intracutaneous, intravenous, intramuscular, intra-articular, intrasynovial, intrasternal, intrathecal, intralesional and intracranial injection or infusion techniques. Administration of antigens in accordance with the methods described herein also may be accomplished by any of the foregoing routes.

According to another embodiment, the invention relates to methods of preventing a food allergy in a subject. These methods include administering therapeutically effective amounts of antigens directly to the subject or administering therapeutically effective amounts of antigens to the subject's mother (e.g., during pregnancy or in a period when she is breastfeeding the subject.) The antigens may (but need not) be administered in the form of the nutritional supplement formulation described above.

In direct administration, therapeutically effective amounts of allergens or potentially-allergenic substances present in one, two, three, four, five, six, seven, or eight of the eight types of foods commonly responsible for food allergies are administered directly to the subject. The subject in this case is preferably a human infant, toddler, or child.

Modes of indirect administration are envisioned. In the first mode, therapeutically effective amounts of allergens present in one, two, three, four, five, six, seven, or eight types of foods commonly responsible for food allergies are administered to a pregnant woman carrying the subject. In one embodiment the allergens are provided as a nutritional supplement formulation, as described herein. Without being bound to any particular theory, the allergens administered may travel via the placenta to the developing embryo or fetus, the immune system of which in turn “learns” that food containing these allergens is “safe” and that no allergic reaction is needed.

In the second mode, therapeutically effective amounts of allergens present in one, two, three, four, five, six, seven or eight allergens or the nutritional supplement is administered to a woman who is in a period of breastfeeding her child or children. Without being bound to any particular theory, the allergens may reach the child via the breast milk. Similar to administration during pregnancy, this method of administration provides early exposure of the subject to allergens from these types of food.

In either of the two above-described modes of indirect administration, the mother may be supplied with one or more supplements or formulations of allergens for use during the treatment regimen of the subject. In another embodiment, the mother may instead be supplied with instructions to consume foods from these groups as part of her diet. Such instructions may include, for example, specific foods or groups of foods to eat, amounts of such foods, and the frequency with which these foods should be consumed.

In any mode of direct or indirect administration, the allergens may be administered together as a cocktail, or may be administered individually. The allergens may be administered on different days, or at different times on the same day. Each allergen may be administered at its own frequency, and in its own quantity. The allergens may be administered as components of an infant formula, for example for bottle feeding or as a substitute or supplement for breastfeeding.

Administration of allergens or a nutritional supplement formulation containing the same may be conducted when the subject is in utero (e.g., from conception until birth, from the fourth week of pregnancy until birth, from the eighth week of pregnancy until birth, from the twelfth week of pregnancy until birth, from the sixteenth week of pregnancy until birth, from the twentieth week of pregnancy until birth, etc.), or from birth toabout four years of age, or from the age of about three months to about three years, or about three months to about two years, or about three months to about eighteen months, or about three months to about twelve months, or about three months to about nine months, or about three months to about six months, or about four months to about two years, or about four months to about eighteen months, or about four months to about twelve months, or about four months to about nine months, or about four months to about six months. Children who are exposed to allergens within this range of ages, but who do not continue to regularly eat the foods containing said allergens, may nonetheless be free of allergy to these foods later in life.

Typically, the antigens administered in accordance with the methods described herein (including when administered in nutritional supplement formulations) will be administered as frequently as about 1 to 5 times per day, or once every other day, or once every third day, or twice weekly, or once weekly, or once every two weeks, or so forth. Where the antigens are administered in the formulations described herein, the amount of active ingredient that may be combined with the carrier materials to produce a single dosage form will vary depending upon the host treated and the particular mode of administration. A typical preparation will contain from about 5% to about 95% allergen as a measure of the total weight of the preparation. Preferably, such preparations contain from about 20% to about 80% allergen.

The nutritional supplement formulations of this invention may be orally administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and solutions and propylene glycol are administered orally, the active ingredient is combined with emulsifying and suspending agents. If desired, certain sweetening and/or flavoring and/or coloring agents may be added. Oral administration of antigens in accordance with the methods described herein also may be accomplished by any of the foregoing dosage forms.

In the methods of preventing food allergies described herein, the therautically effective amounts of each antigen may be expressed in terms of the amount (in weight) of antigen administered or in terms of the amount of antigen present in a given number of servings of the corresponding food. Where applicable, the amount of allergen may also be expressed in allergy units per milliliter (AU/mL) or bioequivalent allergy units (BAU) as established by the American Academy of Allergy, Asthma, and Immunology.

When the allergen or allergens are administered to the mother of the subject, the therapeutically effective amount of each antigen, on a per dose basis, may range from about 10 nanograms to about 500 grams of allergen, or about 100 nanograms to about 100 grams, or about 1 microgram to about 10 grams, or about 10 micrograms to about 1 gram, or about 50 micrograms to about 500 milligrams, or about 100 micrograms to about 100 milligrams, or about 200 micrograms to about 50 milligrams, or about 500 micrograms to about 10 milligrams, or about 1 milligram to about 5 milligrams, or any amount in any of the disclosed ranges. When expressed in terms of servings, a therapeutically effective amount of each antigen is the amount of antigen present in between about 1 serving per week and about 100 servings per week of the corresponding food, between about 2 servings per week and about 50 servings per week of the corresponding food, between about 3 servings per week and about 20 servings per week of the corresponding food, between about 4 servings per week and about 10 servings per week of the corresponding food, or about 5 servings per week of the corresponding food, where the serving size is determined by the “Reference Amounts Customarily Consumed” (RACC) established by the U.S. Food and Drug Administration.

When the allergen is administered to the subject, the therapeutically effective amount of each antigen, on a per dose basis, may range from about 10 picograms to about 1 gram of allergen, or about 100 picograms to about 100 milligrams, or about 100 picograms to about 10 milligrams, or about 1 nanogram to about 1 milligram, or about 10 nanograms to about 500 micrograms, or about 100 nanograms to about 100 micrograms, or about 200 nanograms to about 50 micrograms, or about 500 nanograms to about 40 micrograms, or about 1 microgram to about 35 micrograms, or about 2 micrograms to about 30 micrograms, or about 5 micrograms to about 25 micrograms, or any amount in any of the disclosed ranges. When expressed in terms of servings, a therapeutically effective amount of each antigen is the amount of antigen present in between about 1 serving per month and about 100 servings per month of the corresponding food, between about 2 servings per month and about 50 servings per month of the corresponding food, between about 3 servings per month and about 20 servings per month of the corresponding food, between about 4 servings per month and about 10 servings per month of the corresponding food, or about 5 servings per month of the corresponding food, where the serving size is determined by the “Reference Amounts Customarily Consumed” (RACC) established by the U.S. Food and Drug Administration.

In some cases, dosage levels may be determined by the weight of the subject, the weight of the pregnant or nursing mother, or both. Dosage levels of between about 0.01 and about 100 mg/kg body weight per day of allergen from each type of food, between 0.5 and about 75 mg/kg body weight per day, or between about 1 and 50 mg/kg body weight per day of the active ingredient compound are useful in the prevention and treatment of food allergies. These measures apply to the weight of the individual receiving treatment with the formulation, whether the subject, or the mother of the subject.

As the skilled artisan will appreciate, lower or higher doses than those recited above may be required. Specific dosage and treatment regimens for any particular subject will depend upon a variety of factors, including the activity of the specific antigen employed, the age, body weight, general health status, sex, diet, time of administration, rate of excretion, drug combination, and the patient's disposition to the allergy in question and the judgment of the treating physician.

In one embodiment, the compositions and methods will therefore be useful for controlling, treating or reducing the advancement, severity or effects of food allergies in a subject. In another embodiment, the food allergy is characterized by sensitivity to or allergic reaction to one or more of a peanut, tree nut, milk, soy, wheat, egg, fish, or shellfish.

In order that this invention be more fully understood, the following examples are set forth. These examples are for the purpose of illustration only and are not to be construed as limiting the scope of the invention in any way.

### EXAMPLES

### Example 1

A capsule containing about 100 micrograms of each of a peanut allergen, a tree nut allergen, a milk allergen, a wheat allergen, an egg allergen, a soy allergen, a fish allergen, and a shellfish allergen is provided.

### Example 2

A nursing mother with a three-month-old child is instructed to consume one serving of each of peanuts, tree nuts, cow's milk, wheat, egg, soy, fish, and shellfish at least two times per week. She is to continue observing this dietary rule until the child is six months old, or until the child is weaned, whichever comes first.

### Example 3

A pregnant woman, twelve weeks into her pregnancy, is instructed to consume one serving of each of peanuts, tree nuts, cow's milk, wheat, egg, soy, fish, and shellfish at least two times per week. She is to continue observing this dietary rule until the child is born.

### Example 4

A tablet containing about 100 micrograms of each of a peanut allergen, a tree nut allergen, a milk allergen, a wheat allergen, an egg allergen, a soy allergen, a fish allergen, and a shellfish allergen is provided to a pregnant woman, twelve weeks into her pregnancy. She is instructed to take one table every other day throughout pregnancy until the child is born.

### Example 5

A baby formula is provided. Out of every 30 ounces of liquid formula, 100 micrograms of each of a peanut allergen, a tree nut allergen, a milk allergen, a wheat allergen, an egg allergen, a soy allergen, a fish allergen, and a shellfish allergen are included in the formula.

### Example 6

A liquid solution containing a peanut allergen, a tree nut allergen, a milk allergen, a wheat allergen, an egg allergen, a soy allergen, a fish allergen, and a shellfish allergen in a pharmaceutical carrier and with a flavorant is provided. Each allergen is provided at a concentration of 50 micrograms per milliliter of the solution. A child is administered five milliliters of solution every third day between the time he is three months old and the time is nine months old.

### Example 7: Clinical Study

Supplement Formulations: The Supplement Formulations used in the study contain three antigens selected from the group consisting of a peanut allergen, a milk allergen, a wheat allergen, a tree nut allergen, an egg allergen, a soy allergen, a fish allergen, and a shellfish allergen. Two Supplement Formulations are prepared. The two Supplement Formulations each contain the same three antigens, but may differ in the amounts of the three antigens or in the identities and amounts of excipients present. Supplement Formulation #1 is intended for administration to pregnant and nursing mothers. Supplement Formulation #2 is intended for administration to children between six months of age and one year of age.

Study Design:

| | |
|---|---|
| Study | A cohort of expectant mothers are administered Supplement |
| Arm 1 | Formulation #1 once per day, beginning at the 12^{th} week of |
| | pregnancy and continuing until child birth. After birth, the |
| | children are nourished in accordance with existing guidelines |
| | (see e.g., S. Anvari et al., Evolution of Guidelines on Peanut |
| | Allergy and Peanut Introduction in Infants: A Review, 171 J. |
| | Am. Med. Assoc. 77 (2017)) until the children reach one year |
| | of age. |
| Study | A cohort of expectant mothers are administered Supplement |
| Arm 2 | Formulation #1 once per day, beginning at the 12^{th} week of |
| | pregnancy and continuing until the children reach six months |
| | of age, during which time the children are nourished |
| | exclusively by breastfeeding. After the children reach six |
| | months of age, the children are nourished in accordance with |
| | existing guidelines until the children reach one year of age. |
| Study | A cohort of expectant mothers are administered Supplement |
| Arm 3 | Formulation #1 once per day, beginning at the 12^{th} week of |
| | pregnancy and continuing until the children reach six months |
| | of age, during which time the children are nourished |
| | exclusively by breastfeeding. After the children reach six |
| | months of age, the children are nourished in accordance with |
| | existing guidelines, except that the children also are |
| | administered Supplement Formulation #2 once per day until |
| | the children reach one year of age. |
| Control | A cohort of expectant mothers are nourished in accordance |
| Arm | with existing guidelines from the time that pregnancy is |
| | determined until child birth. After birth, the children are |
| | nourished in accordance with existing guidelines until the |
| | children reach one year of age. |

Analysis:

At one year of age, the children in each arm of the study are tested for allergies to the antigens present in the Supplement Formulations by measurement of the relevant serum IgE levels.

Between one and five years of age, the children are observed by their parents/guardians for the development of allergies to the antigens present in the Supplement Formulations. The parents/guardians complete an annual questionnaire documenting any observed allergies to such antigens.

1. A nutritional supplement formulation comprising therapeutically effective amounts of a first allergen, a second allergen, a third allergen, and a pharmaceutically acceptable carrier.

2. The nutritional supplement formulation of claim 1, wherein the first allergen, the second allergen, and the third allergen are selected from the group consisting of a peanut allergen, a milk allergen, a wheat allergen, a tree nut allergen, an egg allergen, a soy allergen, a fish allergen, and a shellfish allergen.

3. (canceled)

4. (canceled)

5. (canceled)

6. (canceled)

7. (canceled)

8. (canceled)

9. (canceled)

10. (canceled)

11. (canceled)

12. (canceled)

13. A method of preventing food allergies in a subject comprising administering therapeutically effective amounts of a first, second, and third allergen to the subject's mother.

14. The method of claim 13, wherein administering is performed during pregnancy.

15. (canceled)

16. (canceled)

17. (canceled)

18. The method of claim 13, wherein administering is performed during a period in which the mother is breastfeeding.

19. The method of claim 18, wherein the subject is less than twelve months old.

20. (canceled)

21. (canceled)

22. (canceled)

23. The method of claim 13, wherein each of the first, second, and third allergens is administered two times per week.

24. The method of claim 13, wherein each of the first, second, and third allergens is administered orally to the subject's mother.

25. (canceled)

26. (canceled)

27. (canceled)

28. (canceled)

29. The method of claim 13, wherein the subject is substantially free of allergic reaction to the first, second, and third allergens after 10 years.

30. The method of claim 13, wherein the first allergen is administered at 10 nanograms to about 500 grams on a per-dose basis.

31. A method of preventing food allergies in a subject comprising administering therapeutically effective amounts of a first, second, and third allergen to a subject.

32. The method of claim 31, wherein the subject is between birth and about 36 months of age.

33. The method of claim 31, wherein each of the first, second, and third allergens is administered two times per week.

34. The method of claim 31, wherein each of the first, second, and third allergens is administered orally.

35. (canceled)

36. (canceled)

37. (canceled)

38. (canceled)

39. The method of claim 31, wherein the subject is substantially free of allergic reaction to the first, second, and third allergens after 10 years.

40. The method of claim 31, wherein each of the first, second, and third allergens is administered at 10 nanograms to about 500 grams on a per-dose basis.

41. (canceled)

42. (canceled)

43. The method of claim 13, wherein the first, second, and third allergens are selected from the group consisting of a peanut allergen, a milk allergen, a wheat allergen, a tree nut allergen, an egg allergen, a soy allergen, a fish allergen, and a shellfish allergen.

44. The method of claim 31, wherein the first, second, and third allergens are selected from the group consisting of a peanut allergen, a milk allergen, a wheat allergen, a tree nut allergen, an egg allergen, a soy allergen, a fish allergen, and a shellfish allergen.
